(19) United States
(12) Patent Application Publication
(10) Pub. No.: US 2023/0233794 A1
(43) Pub. Date: Jul. 27, 2023

(54) DEEP LEARNING-BASED SLEEP ASSISTANCE SYSTEM THROUGH OPTIMIZATION OF ULTRADIAN RHYTHM

(71) Applicant: Seung Wan ROH, Seongnam-si (KR)

(72) Inventor: Seung Wan ROH, Seongnam-si (KR)

(21) Appl. No.: 18/097,891

(22) Filed: Jan. 17, 2023

(30) Foreign Application Priority Data
Jan. 27, 2022 (KR) 10-2022-0012510
Feb. 23, 2022 (KR) 10-2022-0023311

(51) Int. Cl. A61M 21/02; G05B 13/02

(52) U.S. Cl. CPC A61M 21/02; G05B 13/027; A61M 2021/0027

(57) ABSTRACT

Disclosed herein are a sound sleep assistance apparatus, a sound sleep assistance method, and a sound sleep assistance system. According to an embodiment, there is provided a sound sleep assistance apparatus for assisting the sound sleep of a user by communicating with a sleep pad, the sound sleep assistance apparatus including: a communication interface configured to communicate with the sleep pad that acquires the physiological index information of the user while the user lies down; and a controller configured to determine the sleep stage of the user based on the physiological index information, and to provide a sound source corresponding to the determined sleep stage.

### CROSS-REFERENCE TO RELATED APPLICATION

This application claims the benefit of Korean Patent Application No. 10-2022-0012510 filed on Jan. 27, 2022, and Korean Patent Application No. 10-2022-0023311 filed on Feb. 23, 2022, which are hereby incorporated by reference herein in its entirety.

### BACKGROUND

### 1. Technical Field

The embodiments disclosed herein relate to a sound sleep assistance apparatus, a sound sleep assistance method, and a sound sleep assistance system, and more particularly, to an artificial intelligence-based sound sleep assistance apparatus, an artificial intelligence-based sound sleep assistance method, and an artificial intelligence-based sound sleep assistance system. The embodiments disclosed herein relate to a deep learning-based sleep assistance system through the optimization of an ultradian rhythm.

This study was conducted as a part of the research into the Small and Medium Business Technological Innovation & Development Project (market-responsive type) sponsored by the Korean Ministry of SMEs and Startups and the Korea Technology and Information Promotion Agency for SMEs (TIPA) (TIPA-S3216503) .

### 2. Description of the Related Art

Recently, as interest in well-being has increased, interest in sound sleep has been increasing. Accordingly, the technologies that collect and analyze users’ sleep-related data and help to have sound sleep are classified as one type of technology, and are called sleep technology. However, the conventional sleep technology is problematic in that it is difficult to induce users to continuously use the service and the effect of serviced content is poor. Furthermore, the conventional sleep technology merely monitors physiological changes according to sleep, so that it is not possible to assist users in sleeping sound.

In connection with this, Korean Patent Application Publication No. 10-2019-0064055 entitled “Sound Sleep Induction and Management System Using Sensing Information of Electric Bed” proposes a sound sleep induction and management system, including: a bed driving device configured to detect whether a user snores based on breathing rate and heart rate according to changes in weight caused by the user’s tossing and turning during sleep through sleep pressure sensors capable of detecting the user’s heart rate, breathing rate and weight disposed on one side of the lower part of an electric bed, and to drive massage motors and angle driving motors respectively provided in the backrest and leg portions of the lower part of the bed through a motor controller that operates in conjunction with the motors, thereby preventing snoring, detecting one or more sleep disturbance factors, and providing a sleep environment; and a remote controller device configured to operate in conjunction with the bed driving device via wireless communication such as Bluetooth or ZigBee communication, and to transmit a signal necessary for the driving of the bed to the motor controller of the bed driving device so that the bed driving device can be driven as desired by the user.

This conventional technology is intended to provide a sound sleep environment by preventing snoring. To this end, the bed driving device including various motors must be provided, which requires lots of resources and costs for the sound sleep of a user.

Therefore, there is a demand for a new level of functionality that overcomes the above-described problems and further improves the satisfaction of users who want to sleep sound.

Meanwhile, the above-described background technology corresponds to technical information that has been possessed by the present inventor in order to contrive the present invention or that has been acquired in the process of contriving the present invention, and can not necessarily be regarded as well-known technology that had been known to the public prior to the filing of the present invention.

### SUMMARY

An object of the embodiments disclosed herein is to propose a sound sleep assistance method, a sound sleep assistance apparatus, and a sound sleep assistance system.

An object of the embodiments disclosed herein is to propose a deep learning-based sleep assistance system through the optimization of an ultradian rhythm.

An object of the embodiments disclosed herein is to propose a sound sleep assistance method, a sound sleep assistance apparatus, and a sound sleep assistance system capable of recommending a user-customized lifestyle or providing a customized sound sleep program.

An object of the embodiments disclosed herein is to propose a sound sleep assistance method, a sound sleep assistance apparatus, and a sound sleep assistance system capable of estimating a user’s sleep stage in real time and then recommending a suitable sound source or lifestyle.

An object of the embodiments disclosed herein is to propose a sound sleep assistance method, a sound sleep assistance apparatus, and a sound sleep assistance system capable of estimating a user’s sleep stage and then automatically providing content such as a sound source.

As a technical solution for accomplishing at least any one of the above-described objects, according to an embodiment, there is provided a sound sleep assistance apparatus for assisting the sound sleep of a user by communicating with a sleep pad, the sound sleep assistance apparatus including: a communication interface configured to communicate with the sleep pad that acquires the physiological index information of the user while the user lies down; and a controller configured to determine the sleep stage of the user based on the physiological index information, and to provide a sound source corresponding to the determined sleep stage.

According to another embodiment, there is provided a sound sleep assistance method performed by a sound sleep assistance apparatus, the sound sleep assistance method including: acquiring the physiological index information of a user by communicating with a sleep pad that acquires the physiological index information of the user while the user lies down; determining the sleep stage of the user based on the physiological index information; and providing a sound source corresponding to the determined sleep stage.

According to still another embodiment, there is provided a non-transitory computer-readable storage medium having stored thereon a program that, when executed by a processor, causes the processor to execute a sound sleep assistance method, wherein the sound sleep assistance method includes acquiring the physiological index information of a user by communicating with a sleep pad that acquires the physiological index information of the user while the user lies down, determining the sleep stage of the user based on the physiological index information, and providing a sound source corresponding to the determined sleep stage.

According to still another embodiment, there is provided a computer program that is executed by a sound sleep assistance apparatus and stored in a non-transitory computer-readable storage medium in order to perform a sound sleep assistance method, wherein the sound sleep assistance method includes acquiring the physiological index information of a user by communicating with a sleep pad that acquires the physiological index information of the user while the user lies down, determining the sleep stage of the user based on the physiological index information, and providing a sound source corresponding to the determined sleep stage.

### BRIEF DESCRIPTION OF THE DRAWINGS

The above and other objects, features, and advantages of the present invention will be more clearly understood from the following detailed description taken in conjunction with the accompanying drawings, in which:

FIG. 1 is a diagram illustrating the configuration of a sleep assistance system according to an embodiment disclosed herein;

FIG. 2 is a block diagram illustrating a sleep assistance apparatus according to an embodiment disclosed herein;

FIGS. 3 to 6 are exemplary diagrams illustrating the sleep assistance apparatus according to the embodiment disclosed herein; and

FIG. 7 is a flowchart illustrating a sleep assistance method according to an embodiment disclosed herein.

### DETAILED DESCRIPTION

Various embodiments will be described in detail below with reference to the accompanying drawings. The following embodiments may be modified to and practiced in various different forms. In order to more clearly illustrate features of the embodiments, detailed descriptions of items that are well known to those having ordinary skill in the art to which the following embodiments pertain will be omitted. Furthermore, in the drawings, portions unrelated to descriptions of the embodiments will be omitted. Throughout the specification, like reference symbols will be assigned to like portions.

Throughout the specification, when one component is described as being “connected” to another component, this includes not only a case where the one component is “directly connected” to the other component but also a case where the one component is “connected to the other component with a third component arranged therebetween.” Furthermore, when one portion is described as “including” one component, this does not mean that the portion does not exclude another component but means that the portion may further include another component, unless explicitly described to the contrary.

Embodiments of the present invention will be described in detail below with reference to the accompanying drawings.

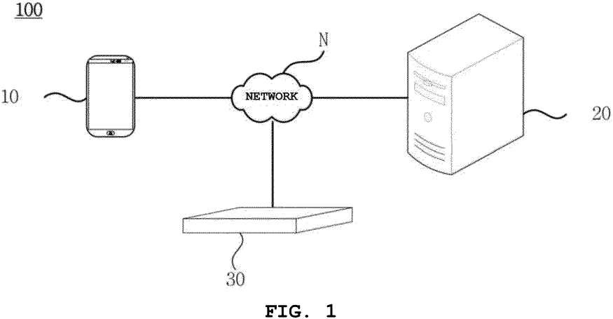

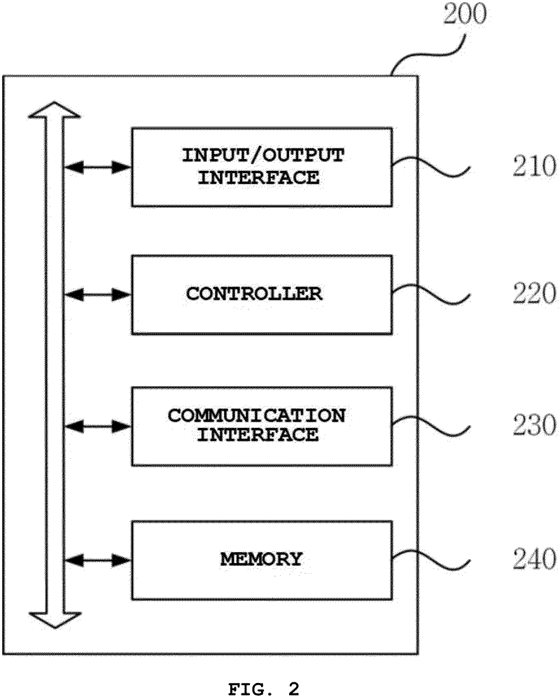

FIG. 1 is a diagram illustrating the configuration of a sleep assistance system 100 according to an embodiment disclosed herein, and FIG. 2 is a block diagram illustrating a sleep assistance apparatus according to an embodiment disclosed herein.

The sleep assistance system 100 may recommend a sleep sound source through synchronization with a user’s ultradian rhythm and also recommend the user’s sleep score and lifestyle based on artificial intelligence.

As shown in FIG. 1, the sleep assistance system 100 may include a sleep assistance apparatus 200 and a sleep pad 30, and the sleep assistance apparatus 200 and the sleep pad 30 may communicate over a network N.

The sleep assistance apparatus 200 may be implemented as an electronic terminal installed with an application capable of interacting with a user, or may be implemented as a server-client system. When the sleep assistance apparatus 200 is implemented as a server-client system, it may include an electronic terminal in which an application for an online service for interaction with a user is installed.

According to an embodiment disclosed herein, the sleep assistance apparatus 200 may be implemented as a server-client system, and may thus be implemented to include a user terminal 10 and a server 20, as shown in FIG. 1.

In this case, the user terminal 10 may be implemented as a computer, a mobile terminal, a television, a wearable device, or the like that can access a remote server or connect with another terminal and a server over a network. In this case, the computer includes, e.g., a notebook, a desktop, a laptop, and the like each equipped with a web browser. The mobile terminal is, e.g., a wireless communication device capable of guaranteeing portability and mobility, and may include all types of handheld wireless communication devices, such as a Personal Communication System (PCS) terminal, a Personal Digital Cellular (PDC) terminal, a Personal Handyphone System (PHS) terminal, a Personal Digital Assistant (PDA), a Global System for Mobile communications (GSM) terminal, an International Mobile Telecommunication (IMT)-2000 terminal, a Code Division Multiple Access (CDMA)-2000 terminal, a W-Code Division Multiple Access (W-CDMA) terminal, a Wireless Broadband (Wibro) Internet terminal, a smartphone, a Mobile Worldwide Interoperability for Microwave Access (mobile WiMAX) terminal, and the like. Furthermore, the television may include an Internet Protocol Television (IPTV), an Internet Television (Internet TV), a terrestrial TV, a cable TV, and the like. Moreover, the wearable device is an information processing device of a type that can be directly worn on a human body, such as a watch, glasses, an accessory, clothing, shoes, or the like, and can access a remote server or connect with another terminal directly or via another information processing device over a network.

In addition, the server 20 may be implemented as a computer capable of communicating with an electronic terminal, having an application for interaction with the manager of the sleep assistance apparatus 200 or a web browser installed therein, over a network, or may be implemented as a cloud computing server. Furthermore, the server 20 may include a storage device capable of storing data, or may store data through a third server.

In this case, according to an embodiment, the server 20 may analyze and learn physiological index information acquired through the sleep pad 30 to be described later, while the user terminal 10 may provide visual and/or auditory content for a user’s sound sleep in real time. For communication with the user terminal 10, the server 20 may perform socket communication, e.g., by converting data to be transmitted to the user terminal 10 into a JSON string and encoding it in Unicode.

Meanwhile, as shown in FIG. 1, the sleep assistance apparatus 200 may communicate with the sleep pad 30 over the network N.

According to an embodiment, the sleep pad 30 may collect physiological index information. In this case, the physiological index information refers to general information for the measurement of the quality of sleep before sleep, during sleep, or upon waking up. For example, the physiological index information may include at least one of heart rate, snoring, and tossing and turning.

The sleep pad 30 is implemented as a mat that can be laid under the user when the user lies down. In the state in which the user is lying on the sleep pad 30, the physiological index information of the user, including at least one of the user’s heart rate, snoring, and tossing and turning, may be acquired. To this end, various types of sensors for collecting the physiological index information of the user may be included in the sleep pad 30. For example, a piezoelectric film sensor may be included in the sleep pad 30. The piezoelectric film sensor may be combined with various other sensors, and is fabricated in the form of a film, so that the piezoelectric film sensor is convenient to fabricate and does not cause the feeling of irritation caused by a foreign object when placed in the sleep pad 30.

For example, the sleep pad 30 may generate the user’s tossing and turning information by collecting the user’s movement on the piezoelectric sensor. Furthermore, the sleep pad 30 may detect the user’s heart beat by detecting the user’s arterial quiver through the piezoelectric sensor and calculate the user’s heart rate (bpm) by counting heart rate. Furthermore, the sleep pad 30 may generate snoring information by converting the energy signal of the output signal of the piezoelectric sensor into a decibel signal and continuously detecting the duration and intensity of a snoring event, which is a sound event, from the decibel signal. The physiological index information generated as described above may be transmitted to the sleep assistance apparatus 200.

According to an embodiment, the sleep pad 30 may transmit the physiological index information, collected before sleep, during sleep, and upon waking up, to the sleep assistance apparatus 20, and the sleep assistance apparatus 20 may determine the sleep stage of the user based on the physiological index information and provide a sound source corresponding to the determined sleep stage. For example, the sleep pad 30 may transmit collected physiological index information to the server 20, and the server 20 may provide a sound source, determined based on the physiological index information, to the user through the user terminal 10.

Meanwhile, according to an embodiment, the sleep assistance apparatus 200 may include an input/output interface 210, a controller 220, a communication interface 230, and memory 240.

The input/output interface 210 may include an input interface configured to receive input from the user, and an output interface configured to display information such as the result of the performance of a task or the status of the sleep assistance apparatus 200 or sleep assistance system 100. For example, the input/output interface 210 may include an operation panel configured to receive input from the user and a display panel configured to display screens.

More specifically, the input interface may include various types of devices capable of receiving user input such as a keyboard, physical buttons, a touch screen, a camera, a microphone, and/or the like. Furthermore, the output interface may include a display panel, a speaker and/or the like. However, the input/output interface 210 is not limited thereto, and may include various types of components capable of supporting input/output.

The controller 220 controls the overall operation of the sleep assistance apparatus 200, and may include a processor such as a graphics processing unit (CPU) or a central processing unit (GPU). The controller 220 may control other components included in the sleep assistance apparatus 200 to perform operations corresponding to user inputs received through the input/output interface 210.

For example, the controller 220 may execute a program stored in the memory 240, may read a file stored in the memory 240, or may store a new file in the memory 240. This controller 220 will be described in more detail later.

Meanwhile, according to an embodiment, the communication interface 230 may perform wired/wireless communication with another device or a network. To this end, the communication interface 230 may include a communication module configured to support at least one of various wired/wireless communication methods. For example, the communication module may be implemented in the form of a chipset.

The wireless communication supported by the communication interface 230 may be, e.g., Wireless Fidelity (Wi-Fi), Wi-Fi Direct, Bluetooth, Ultra-Wide Band (UWB), or Near Field Communication (NFC). Furthermore, the wired communication supported by the communication interface 230 may be, e.g., Universal Serial Bus (USB), or High Definition Multimedia Interface (HDMI).

According to an embodiment, the communication interface 230 may acquire physiological index information from the sleep pad 30 while communicating with the sleep pad 30.

Meanwhile, according to an embodiment, various types of data such as a file, an application, and a program may be installed and stored in the memory 240. The controller 220 may access and use data stored in the memory 240, or may store new data in the memory 240. Furthermore, the controller 220 may execute a program installed in the memory 240. Referring to FIG. 2, a program for performing a sleep assistance method may be installed in the memory 240.

According to an embodiment, when receiving an input, requesting a sleep assistance method, from the user through the input/output interface 210, the controller 220 performs the sleep assistance method by executing a program stored in the memory 240.

The controller 220 will be described in more detail below.

According to an embodiment, the controller 220 may determine a user’s sleep stage by determining a sleep state based on physiological index information.

In this case, the controller 220 may first determine whether the user is taking a posture for sleeping prior to determining the user’s sleeping state. In other words, there may be a case where the user simply lies down not to sleep, but to rest. In order to reduce the waste of device resources, it may first be determined whether the user is lying down to take sleep, and then a sleep state may be determined.

According to an embodiment, the controller 220 may detect at least one of a user’s input and the execution of an installed application and then trigger the determination of the user’s sleep state. For example, when the user’s input is successively made to the user terminal 10 used as one component of the sleep assistance apparatus 200, it may be determined that the user is not ready to sleep. Furthermore, for example, when an application installed in the user terminal 10 is executed and an event occurs in the application, it may be determined that the user is not ready to sleep for a predetermined period from the time when the event occurs. Furthermore, for example, when a user input event occurs for an application installed on the user terminal 10, it is determined that the user is not ready to sleep. When an additional input event does not occur when a predetermined time has elapsed since the occurrence of the former event, it may be determined that the user is ready to sleep, and a process for determining the user’s sleep state may be triggered. Accordingly, for example, even when the controller 220 acquires the user’s physiological index information as the sleep pad transmits the physiological index information, it may be determined whether the user is ready to sleep. When a process for determining a sleep state is triggered in response to the result of this determination, the user’s sleep stage may be determined based on the physiological index information.

Meanwhile, sleep consists of non-REM sleep and REM sleep. While a person sleeps, there are shown changes in which non-REM sleep and REM sleep are alternated at cycles of about 90 to 120 minutes, which is called an ultradian rhythm. Accordingly, when a person sleeps for eight hours, which are recommended sleep hours, the ultradian rhythm is repeated four to five times, and the ultradian rhythm needs to be stabilized for a sound sleep. When sleep is managed, it is necessary to think about what to do to sleep deeply in first non-REM sleep. When a person can sleep deeply in this stage, the following sleep rhythm is also stabilized, and thus the autonomic nerves and hormones function smoothly, so that the efficiency of activities the next day increases.

TABLE 1

| | | |
|---|---|---|
| Sleep State: Brain Waves | | Definition |
| Awake: alpha, 8 to 13 Hz | | This is a trance state, and corresponds to an interval similar to that of a hypnotic state. This is a stage in which the eyes are opened even at a slight sound. When the eyes are opened in this stage, wake-up quality is highest. |
| REM: theta, 4 to 7 Hz | | This appears several times during sleep, |
| (Rapid eye movement sleep) | | usually more often in the second half of sleep. In this stage, dreaming occurs mainly, and the eyes move rapidly in various directions. Furthermore, the heart rate increases and breathing becomes more irregular. |
| Non-REM: delta, 0.5 to 3 Hz | Light sleep (N-REM 1) | This is an interval in which a person enters sleep in earnest as his or her body relaxes. Usually, this stage begins within a few minutes after going to sleep. In this case, breathing and heart rate decrease, and there occurs an action that restores the fatigued mind. |
| | Deep sleep (N-REM 2) | This is a sleep pattern that usually appears a few hours after falling asleep. In this stage, breathing slows, muscles relax, and heart rate becomes constant. In this case, there occurs an action that helps to improve the body’s recovery, memory, and immunity. |

As described in Table 1 above, sleep states include awake, REM, light sleep, and deep sleep states. The controller 220 estimates the user’s sleep state and also provides content based on the fact that, when the resonance between the brain waves generated for each sleep state and surrounding environmental factors is induced, the ultradian rhythm is stabilized, and thus the user’s sound sleep can be induced.

The controller 220 may determine the current sleep state of the user by using a trained deep learning model. The deep learning model may be VGGNet, but is not limited thereto. The deep learning model may also be implemented as a deep learning model such as ResNet or MobileNet.

According to an embodiment, the controller 220 may visualize physiological index information in order to train the deep learning model, and may train the deep learning model by using the visualized physiological index information and sleep states as input values. For example, the controller 220 may train the deep learning model by using visualized heart rate data and sleep states as input values. Accordingly, when features are extracted using a sliding window algorithm, one piece of learning data may be generated by grouping a predetermined number of pieces of heart rate data and converting them into a matrix on the assumption that a plurality of pieces of heart rate data are present for each minute, and a matrix may be generated by selecting a window in which grouping target data is selected with a stride of 1 minute and then sliding it. The size of the window may be 25, and thus heart rate data may be converted into a 5*5 image accordingly. The controller 220 may train a deep learning model by using the features extracted as described above and sleep states as input values.

In addition, the controller 220 may infer the user’s sleep state by using a deep learning model trained by visualizing the user’s heart rate data.

Meanwhile, according to an embodiment, the controller 220 may determine content to be provided to the user based on the user’s sleep stage.

In connection with this, the sleep stage refers to information when transition is made from one sleep state to another sleep state, and the controller 220 may provide optimal content for each sleep stage. For example, as the sleep state transitions from one state to another state, content may be changed for each of the sleep stages. Through this, the controller 220 may not provide, e.g., a fixed frequency throughout sleep, but may adaptively change and provide a sound source during each sleep state or while the sleep state changes.

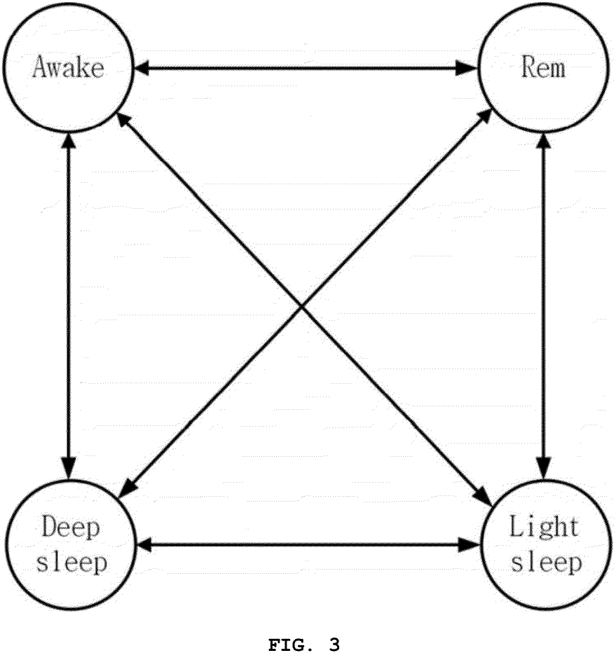

For example, sleep states may be classified into four types. In the case of the transition from one state to a subsequent state, classification into a total of 16 sleep stages may be made as listed in Table 2 below. In connection with this, FIG. 3 is an exemplary diagram illustrating the sleep assistance apparatus according to the embodiment disclosed herein. As shown in FIG. 3, the individual sleep stages may be represented by a perfect direction graph (PDG).

TABLE 2

Sleep Stage (current state ➔ state after 10 minutes)
Awake
Awake ➔ Awake
Awake ➔ Light sleep
REM ➔ Awake
REM ➔ Awake (10 minutes before the alarm)
REM ➔ REM
REM ➔ REM (10 minutes before the alarm)
REM ➔ Light sleep
REM ➔ Light sleep (10 minutes before the alarm)
Light sleep ➔ Light sleep
Light sleep ➔ REM
Light sleep ➔ REM (10 minutes before the alarm)
Light sleep ➔ Deep sleep
Light sleep ➔ Awake
Deep sleep ➔ Deep sleep
Deep sleep ➔ Light sleep
Deep sleep ➔ Awake

The controller 220 may provide content set for each of the sleep stages. To this end, the controller 220 may identify the current sleep state of the user at predetermined cycles and determine a sleep stage according to a previous sleep state and a current sleep state. For example, every 10 minutes, the controller 220 may identify the current sleep state of the user, determine a sleep stage, and provide content suitable for the determined sleep stage.

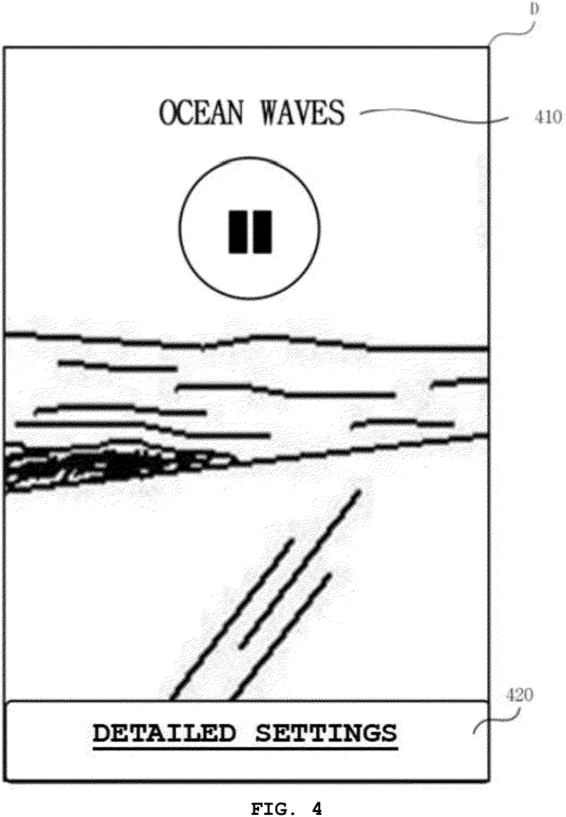

In this case, the content may include a sound source, Autonomous Sensory Meridian Response (ASMR) content, a visual image, or a video. In connection with this, FIG. 4 is an exemplary diagram illustrating the sleep assistance apparatus according to the embodiment disclosed herein. As shown in FIG. 4, the title 410 of a sound source being played back may be displayed along with a visual image through the screen D of the user terminal 10, and a tab 420 for checking detailed information about the sound source may also be provided.

According to an embodiment, the content may be preset for each of the sleep stages. In other words, ASMR content to be played back is set for each of the sleep stages shown in Table 2. When the user reaches a specific sleep stage, the controller 220 may play back ASMR content matched to the corresponding sleep stage.

In addition, a plurality of pieces of content may be matched to each of the sleep stages. In the case where the user’s sleep state is maintained or does not transition to an expected sleep state by one piece of content when the one piece of content is provided, another piece of content may be provided. For example, when a plurality of sound sources corresponding to each of the sleep stages are present, the corresponding sound sources are sequentially set, and it is determined that a sound source played back the most recently is not suitable, the controller 220 may set a next-ranked sound source. In other words, when a sound source to be provided first is present among a plurality of sound sources for each of the sleep stages and also it is determined that the first sound source is not suitable, a candidate sound source may be provided to replace the first sound source, and generally, a sound source ranked next may be provided. Accordingly, when a current sleep state in a corresponding sleep stage is changed earlier or later than expected during a predetermined period of time during which a sound source corresponding to the sleep stage is provided, the sound source of the corresponding sleep stage may be changed from the first sound source to a next-ranked sound source and also the next-ranked sound source may be provided to the user in the future. Furthermore, when the current sleep state is changed earlier or later by the second-ranked sound source, a third-ranked sound source may be provided to the corresponding user in the future.

Furthermore, according to another embodiment, the content may be determined using a deep learning model. For example, the controller 220 may output and provide content suitable for each of the sleep stages by using a deep learning model trained by selecting content, having received feedback of a predetermined reference value or more, from the feedback on content provided for the sleep stage and receiving the selected content and the sleep stage as input. In this case, for example, the deep learning model may be a deep learning model formed by obtaining feedback from a plurality of users and then being trained with content, selected based on the feedback of the plurality of users, and sleep stages. Alternatively, the deep learning model may be a deep learning model formed by obtaining feedback from a user who will be provided with content for a predetermined period of time and then being trained with selected content and sleep stages.

Through this, the controller 220 may analyze the current sleep stage of the user in real time and provide content such as a sound source that can resonate with brain waves generated in the current sleep stage, thereby rapidly stabilizing the user’s ultradian rhythm and thus maintaining an optimal sleep condition for each sleep stage.

Meanwhile, according to an embodiment, the controller 220 may generate a sleep score based on the user’s physiological index information and a corresponding sleep state.

In addition, the controller 220 may generate a sleep score based on the user’s physiological index information, sleep pattern, and wake-up quality. Furthermore, the controller 220 may provide the generated sleep score to the user, and may provide the generated sleep score after determining that the user wakes up.

For example, the controller 220 may calculate a heart rate score, a snoring score, and a tossing and turning score as physiological index information, may calculate a sleep pattern score by analyzing a sleep state having occurred during the user’s sleep, and may generate a wake-up quality score.

When there are a total of four sleep states, the controller 220 may calculate a sleep pattern score based on the ratio of each of the sleep states to the total sleep time. In this case, the controller 220 may calculate a sleep pattern score by identifying the difference between the ratio of the user’s sleep state and a reference ratio and then adding or subtracting a score according to the identified difference. In this case, the reference ratio may be preset, or may be determined by analyzing the sleep of a plurality of users and then calculating the average of the ratios of the sleep states to the total sleep time. Accordingly, for example, as described in Table 3 below, there are reference ratios that are occupied by the respective sleep states. In the case of an ‘awake’ sleep state, a perfect score of 100 is not changed when the ‘awake’ sleep state occupies 10% of the total sleep time. One point may be subtracted from the perfect score for every 2% increase in the ‘awake’ state.

TABLE 3

| | |
|---|---|
| Sleep Pattern | Score (perfect score : 100 points) |
| Awake | standard range: good up to 10%; one point is subtracted for every +2% |
| REM | standard range: good up to 17 to 23%; one point is subtracted for every error range of ± 2% outside the standard range |
| Light | standard range: good up to 45 to 55%; one point is subtracted for every error range of ± 2% outside the standard range |
| Deep | standard range: good up to 13 to 17%; one point is subtracted for every error range of ± 2% outside the standard range |

In addition, the controller 220 may calculate a sleep quality score by analyzing a sleep state for a predetermined period of time from the user’s wake-up time.

For example, as shown in Table 4 below, when the sleep state before waking up is ‘awake,’ the wake-up quality score may be set to a perfect score. In contrast, when the sleep state before waking up is ‘deep sleep,’ the wake-up quality score may be set to 0 points.

TABLE 4

| Wake-up Quality | Score (perfect score: 100 points) |
|---|---|
| Awake ➔ Waking up | 100 |
| REM ➔ Waking up | 80 |
| Light sleep ➔ Waking up | 40 |
| Deep sleep ➔ Waking up | 0 |

In addition, the controller 220 may calculate a physiological index score by analyzing physiological index information such as a heart rate score, a snoring score, and a tossing and turning score.

As described in Table 5 below, the heart rate score may be calculated by subtracting five points from a perfect score of 100 points whenever the user’s average sleeping heart rate during the overall sleeping time is higher or lower than the standard range of the heart rate by a predetermined value (e.g., 2). Accordingly, for example, when it is determined that the average value of the heart rate of the user is 43.5, the controller 220 may determine the heart rate score to be 90 points by subtracting 10 points from the perfect score of 100 points.

TABLE 5

| | |
|---|---|
| Heart Rate | Score (perfect score: 100 points) |
| Average Heart Rate | standard range: 47.5%; five points are subtracted for every error range of ± 2% outside the standard range |

In addition, the controller 220 may count cases where snoring intensity data indicative of snoring intensity has a value larger than a predetermined value and then calculate the snoring score according to the counted number of cases. In other words, the controller 220 may calculate the snoring score by subtracting a predetermined score from a perfect score in proportion to the counted number of cases.

For example, as described in Table 6 below, when the number of cases where the snoring intensity data exceeds, e.g., 80 is counted and the counted number of cases exceeds 10, the controller 220 may calculate the user’s snoring score as 70 points.

TABLE 6

| Snoring | Score (perfect score: 100 points) |
|---|---|
| The number of Cases of Snoring Intensity Exceeding 80 < 1 | 100 |
| The number of Cases of Snoring Intensity Exceeding 80 < 5 | 90 |
| The number of Cases of Snoring Intensity Exceeding 80 < 10 | 80 |
| The number of Cases of Snoring Intensity Exceeding 80 > = 10 | 70 |

In addition, the controller 220 may count cases where tossing and turning intensity data indicative of tossing and turning intensity during sleep is larger than a predetermined value, and may calculate a tossing and turning score according to the counted number of cases. In other words, the controller 220 may calculate the tossing and turning score by subtracting a predetermined score from a perfect score in proportion to the counted number of cases.

For example, as described in Table 7 below, when cases where tossing and turning intensity data exceeds, e.g., 120 are counted and the counted number of cases is less than 30, a perfect score is assigned. In contrast, when the counted number of cases is smaller than 40 and larger than 35, the tossing and turning score may be calculated as 80 points.

TABLE 7

| Tossing and Turning | Score (perfect score: 100 points) |
|---|---|
| The number of Cases of Tossing and Turning Intensity Exceeding 120 < 30 | 100 |
| The number of Cases of Tossing and Turning Intensity Exceeding 120 < 35 | 90 |
| The number of Cases of Tossing and Turning Intensity Exceeding 120 < 40 | 80 |
| The number of Cases of Tossing and Turning Intensity Exceeding 120 > = 40 | 70 |

The controller 220 may calculate the sleep score based on the physiological index score including at least one of the sleep pattern score, the wake-up quality score, the heart rate score, the snoring score, and the tossing and turning score calculated as described above.

For example, the controller 220 may calculate the total or average value of the sleep pattern score, the wake-up quality score, and the physiological index score as the sleep score. Furthermore, for example, the controller 220 may calculate the sleep score by setting weights for respective scores. The score obtained by summing 70% of the sleep pattern score, 10% of each of the wake-up quality and heart rate scores, and 5% of each of the snoring and tossing and turning scores may be calculated as the sleep score.

In addition, when providing the sleep score as well as various types of scores constituting the sleep score, the controller 220 may plot the corresponding scores on graphs and then provide the graphs. Furthermore, the controller 220 may convert the sleep score as well as various types of scores constituting the sleep score into statistics and then provide the statistics for each period. Through this, the user may identify and deal with one or more factors and/or problems interfering with sleep compared to the daily sleep score.

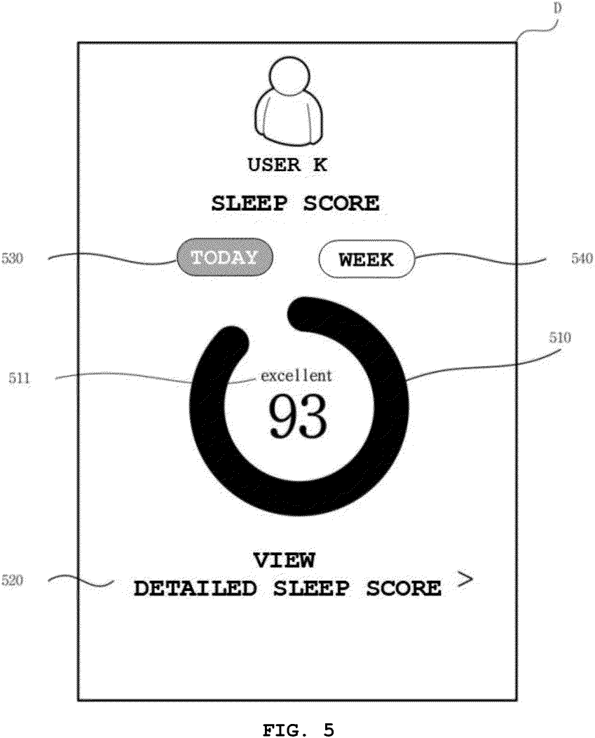

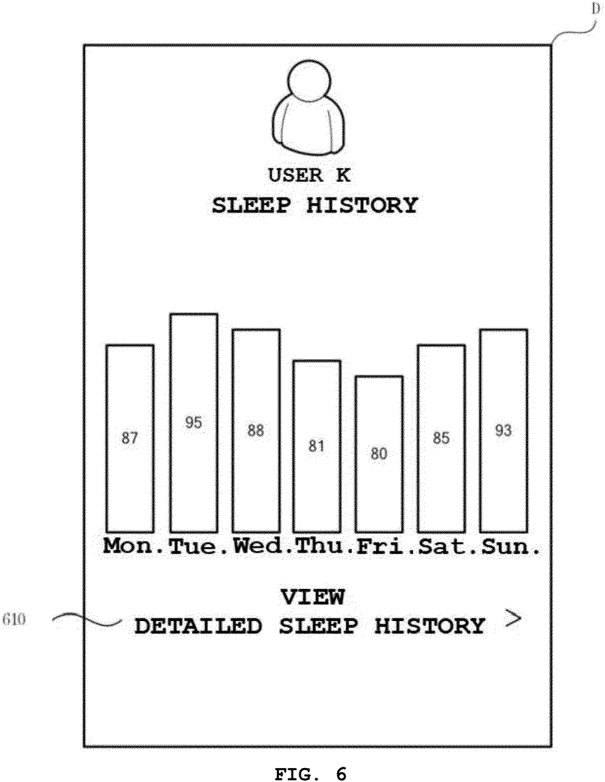

In connection with this, FIGS. 5 and 6 are exemplary diagrams illustrating a sleep assistance apparatus 200 according to an embodiment disclosed herein, which are displayed through the screen D of the user terminal 10.

As shown in FIG. 5, the controller 220 may display the sleep score 510 calculated as described above, and may provide a general review 511, evaluating the sleep score 510, together with the sleep score 510. In addition, the controller 220 may provide a tab 520 through which the sleep score can be inquired about in detail. When the tab 520 is selected by the user, the controller 220 may provide a physiological index score including at least one of various scores constituting a sleep score, i.e., a sleep pattern score, a wake-up quality score, a heart rate score, a snoring score, and a tossing and turning score, so that the user may determine one or more sections for each of which a low score is received. Furthermore, the controller 220 may provide a user’s sleep score on a daily basis, and may also provide a sleep score for today 530 or a sleep score for a week 540 according to the user’s request. The controller 220 may display sleep scores for respective days of the week 540, as shown in FIG. 6. Moreover, the controller 220 may provide a tab 610 through which sleep scores for respective days of the week can be inquired about in detail, and may provide a physiological index score including at least one of various types of scores used to calculate a sleep score for each day of the week, i.e., a sleep pattern score, a wake-up quality score, a heart rate score, a snoring score, and a tossing and turning score.

Through this, users may easily and intuitively receive information on how well he or she slept, what problem he or she had during sleep, and what improvement needs to be made in the future.

Meanwhile, according to an embodiment, the controller 220 may recommend a lifestyle.

According to an embodiment, the controller 220 may recommend a lifestyle based on the sleep score. For example, when it is detected that the snoring score constituting part of the user’s sleep score decreases, the controller 220 may provide information about a lifestyle descriptive of an exercise or therapy for reducing snoring.

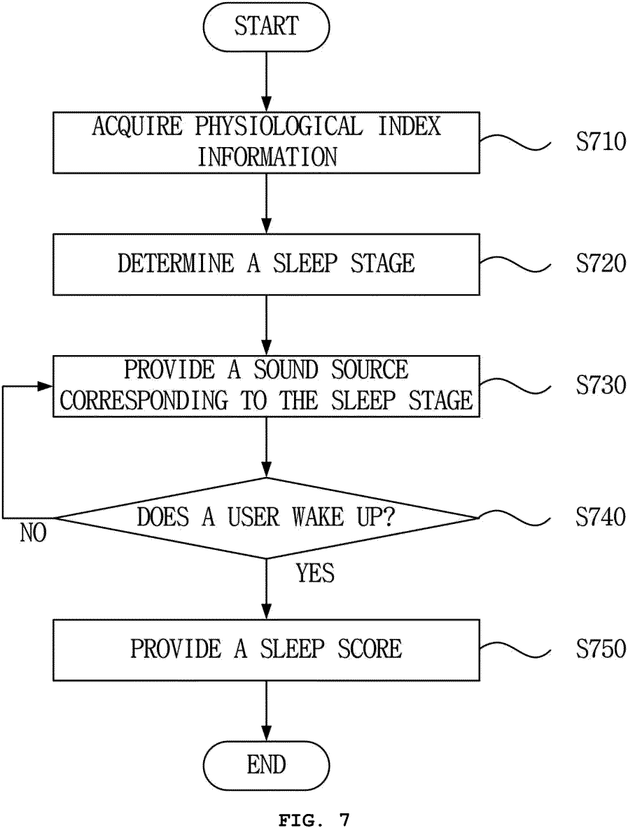

Meanwhile, FIG. 7 is a flowchart illustrating a sleep assistance method according to an embodiment disclosed herein.

The sleep assistance method according to the embodiment shown in FIG. 7 includes steps that are processed in a time-series manner by the sleep assistance apparatus 200 shown in FIG. 7. Accordingly, the descriptions that are omitted below but have been given above in conjunction with the sleep assistance apparatus 200, which is shown in FIGS. 1 to 6, may also be applied to the sleep assistance method, which is shown in FIG. 7.

As shown in FIG. 7, the sleep assistance apparatus 200 may acquire physiological index information by communicating with a sleep pad that acquires the physiological index information of a user while the user is lying down in step S710.

In addition, the sleep assistance apparatus 200 may determine the sleep stage of the user based on the physiological index information.

For example, the sleep assistance apparatus 200 may determine the user’s sleep state based on the physiological index information, and may determine the user’s sleep stage by analyzing the transition from one sleep state to another sleep state. Furthermore, for example, the sleep assistance apparatus 200 may determine the user’s sleep state using a trained deep learning model. More specifically, as an example, physiological index information may be input to the trained deep learning model, and then output information may be predicted as the user’s sleep state.

In addition, the sleep assistance apparatus 200 may provide a sound source corresponding to the determined sleep stage in step S730.

Meanwhile, when the sleep assistance apparatus 200 determines that a sleep state has changed due to the user’s waking up in step S740, the sleep score of the user may be calculated and provided in step S750. For example, the sleep assistance apparatus 200 may calculate a sleep score based on the physiological index information and sleep state of the user. Furthermore, for example, the sleep assistance apparatus 200 may calculate the sleep score based on a physiological index score based on the analysis of the physiological index information of the user, a sleep pattern score based on the analysis of a sleep state, and a wake-up quality score based on the analysis of a sleep state for a predetermined period of time based from the user’s wake-up time.

The term “unit” used in the above-described embodiments means software or a hardware component such as a field-programmable gate array (FPGA) or application-specific integrated circuit (ASIC), and a “unit” performs a specific role. However, a “unit” is not limited to software or hardware. A “unit” may be configured to be present in an addressable storage medium, and also may be configured to run one or more processors. Accordingly, as an example, a “unit” includes components, such as software components, object-oriented software components, class components and task components, processes, functions, attributes, procedures, subroutines, segments in program code, drivers, firmware, microcode, circuits, data, a database, data structures, tables, arrays, and variables.

Components and a function provided in “unit(s)” may be coupled to a smaller number of components and “unit(s)” or divided into a larger number of components and “unit(s).”

In addition, components and “unit(s)” may be implemented to run one or more central processing units (CPUs) in a device or secure multimedia card.

The sleep assistance method according to the embodiments described above may be implemented in the form of a computer-readable medium that stores instructions and data that can be executed by a computer. In this case, the instructions and the data may be stored in the form of program code, and may generate a predetermined program module and perform a predetermined operation when executed by a processor. Furthermore, the computer-readable medium may be any type of available medium that can be accessed by a computer, and may include volatile, non-volatile, separable and non-separable media. Furthermore, the computer-readable medium may be a computer storage medium. The computer storage medium may include all volatile, non-volatile, separable and non-separable media that store information, such as computer-readable instructions, a data structure, a program module, or other data, and that are implemented using any method or technology. For example, the computer storage medium may be a magnetic storage medium such as an HDD, an SSD, or the like, an optical storage medium such as a CD, a DVD, a Blu-ray disk or the like, or memory included in a server that can be accessed over a network.

Furthermore, the sleep assistance method according to the embodiments described above may be implemented as a computer program (or a computer program product) including computer-executable instructions. The computer program includes programmable machine instructions that are processed by a processor, and may be implemented as a high-level programming language, an object-oriented programming language, an assembly language, a machine language, or the like. Furthermore, the computer program may be stored in a tangible computer-readable storage medium (for example, memory, a hard disk, a magnetic/optical medium, a solid-state drive (SSD), or the like).

Accordingly, the sleep assistance method according to the embodiments described above may be implemented in such a manner that the above-described computer program is executed by a computing apparatus. The computing apparatus may include at least some of a processor, memory, a storage device, a high-speed interface connected to memory and a high-speed expansion port, and a low-speed interface connected to a low-speed bus and a storage device. These individual components are connected using various buses, and may be mounted on a common motherboard or using another appropriate method.

In this case, the processor may process instructions within a computing apparatus. An example of the instructions is instructions which are stored in memory or a storage device in order to display graphic information for providing a Graphic User Interface (GUI) onto an external input/output device, such as a display connected to a high-speed interface. As another embodiment, a plurality of processors and/or a plurality of buses may be appropriately used along with a plurality of pieces of memory. Furthermore, the processor may be implemented as a chipset composed of chips including a plurality of independent analog and/or digital processors.

Furthermore, the memory stores information within a computing apparatus. As an example, the memory may include a volatile memory unit or a set of the volatile memory units. As another example, the memory may include a non-volatile memory unit or a set of the non-volatile memory units. Furthermore, the memory may be another type of computer-readable medium, such as a magnetic or optical disk.

In addition, the storage device may provide a large storage space to the computing apparatus. The storage device may be a computer-readable medium, or may be a configuration including such a computer-readable medium. For example, the storage device may also include devices within a storage area network (SAN) or other elements, and may be a floppy disk device, a hard disk device, an optical disk device, a tape device, flash memory, or a similar semiconductor memory device or array.

According to any one of the above-described solutions, there may be proposed the sound sleep assistance method, the sound sleep assistance apparatus, and the sound sleep assistance system.

According to any one of the above-described solutions, there may be proposed the deep learning-based sleep assistance system through the optimization of an ultradian rhythm.

According to any one of the above-described solutions, there may be proposed the sound sleep assistance method, the sound sleep assistance apparatus, and the sound sleep assistance system capable of recommending a user-customized lifestyle or providing a customized sound sleep program.

According to any one of the above-described solutions, there may be proposed the sound sleep assistance method, the sound sleep assistance apparatus, and the sound sleep assistance system capable of estimating a user’s sleep stage in real time and then recommending a suitable sound source or lifestyle. Through this, it may be possible to provide content suitable for immediately before sleep, during sleep, and waking up after sleep, thereby improving the effectiveness of the content. Furthermore, it may be possible to overcome the problem in which the quality of a sleep induction sound source is deteriorated by providing the sleep analysis results of a previous day after waking up by using a conventional technology.

According to any one of the above-described solutions, there may be proposed the sound sleep assistance method, the sound sleep assistance apparatus, and the sound sleep assistance system capable of estimating a user’s sleep stage and then automatically providing content such as a sound source. Since the user’s separate effort is not required, it may be possible to allow the user to continue to use the service according to the invention described herein and to maintain the retention of the service.

The effects that can be obtained by the embodiments disclosed herein are not limited to the effects described above, and other effects not described above will be clearly understood by those of ordinary skill in the art, to which the present invention pertains, from the foregoing description.

The above-described embodiments are intended for illustrative purposes. It will be understood that those having ordinary knowledge in the art to which the present invention pertains can easily make modifications and variations without changing the technical spirit and essential features of the present invention. Therefore, the above-described embodiments are illustrative and are not limitative in all aspects. For example, each component described as being in a single form may be practiced in a distributed form. In the same manner, components described as being in a distributed form may be practiced in an integrated form.

The scope of protection pursued through the present specification should be defined by the attached claims, rather than the detailed description. All modifications and variations which can be derived from the meanings, scopes and equivalents of the claims should be construed as falling within the scope of the present invention.

What is claimed is:

1. A sound sleep assistance apparatus for assisting sound sleep of a user by communicating with a sleep pad, the sound sleep assistance apparatus comprising:
a communication interface configured to communicate with the sleep pad that acquires physiological index information of the user while the user lies down; and
a controller configured to determine a sleep stage of the user based on the physiological index information, and to provide a sound source corresponding to the determined sleep stage.

2. The sound sleep assistance apparatus of claim 1, wherein the controller determines a sleep state of the user based on the physiological index information, and determines the sleep stage of the user by analyzing transition from one sleep state to another sleep state.

3. The sound sleep assistance apparatus of claim 2, wherein the controller determines the sleep state of the user by using a trained deep learning model.

4. The sound sleep assistance apparatus of claim 1, wherein the controller calculates a sleep score based on the physiological index information of the user and a sleep state of the user.

5. The sound sleep assistance apparatus of claim 4, wherein the controller calculates the sleep score based on a physiological index score based on an analysis of the physiological index information of the user, a sleep pattern score based on an analysis of the sleep state, and a wake-up quality score based on an analysis of a sleep state for a predetermined period of time from the user’s wake-up time.

6. The sound sleep assistance apparatus of claim 1, wherein the controller triggers the determination of the sleep stage in response to detection of at least one of the user’s input and execution of an installed application.

7. The sound sleep assistance apparatus of claim 1, wherein the controller provides the sound source, and provides a candidate sound source for the former sound source when a sleep state in the sleep stage is not maintained for a predicted period of time after the provision of the former sound source.

8. A sound sleep assistance system for assisting sound sleep of a user, the sound sleep assistance system comprising:
a sleep pad configured to acquire physiological index information of the user while the user lies down; and
a sound sleep assistance apparatus configured to determine a sleep stage of the user based on the physiological index information, and to provide a sound source corresponding to the determined sleep stage.

9. The sound sleep assistance system of claim 8, wherein the sleep pad comprises a piezoelectric film sensor.

10. A sound sleep assistance method performed by a sound sleep assistance apparatus, the sound sleep assistance method comprising:
acquiring physiological index information of a user by communicating with a sleep pad that acquires the physiological index information of the user while the user lies down;
determining a sleep stage of the user based on the physiological index information; and
providing a sound source corresponding to the determined sleep stage.

11. The sound sleep assistance method of claim 10, wherein determining the sleep stage of the user comprises determining a sleep state of the user based on the physiological index information and determining the sleep stage of the user by analyzing transition from one sleep state to another sleep state.

12. The sound sleep assistance method of claim 11, wherein determining the sleep stage of the user comprises determining the sleep state of the user by using a trained deep learning model.

13. The sound sleep assistance method of claim 10, further comprising calculating a sleep score based on the physiological index information of the user and a sleep state of the user.

14. The sound sleep assistance method of claim 13, wherein calculating the sleep score comprises calculating the sleep score based on a physiological index score based on an analysis of the physiological index information of the user, a sleep pattern score based on an analysis of the sleep state, and a wake-up quality score based on an analysis of a sleep state for a predetermined period of time from the user’s wake-up time.

15. A non-transitory computer-readable storage medium having stored thereon a program that, when executed by a processor, causes the processor to execute the sound sleep assistance method set forth in claim 10.

16. A computer program that is executed by a sound sleep assistance apparatus and stored in a non-transitory computer-readable storage medium in order to perform the sound sleep assistance method set forth in claim 10.
